(19) United States
(12) Plant Patent Application Publication
(10) Pub. No.: US 2009/0064384 P1
(43) Pub. Date: Mar. 5, 2009

(54) POINSETTIA PLANT NAMED 'FISMARS LIPINK'

(76) Inventor: Katharina Zerr, Hohr-Grenzhausen (DE)

Correspondence Address: JONDLE & ASSOCIATES P.C., 858 HAPPY CANYON ROAD SUITE 230, CASTLE ROCK, CO, 80108, US

(21) Appl. No.: 11/899,075

(22) Filed: Sep. 4, 2007

(51) Int. Cl. A01H 5/00

(52) U.S. Cl. PLT/306

(57) ABSTRACT

A Poinsettia cultivar particularly distinguished by light pink bracts, medium-sized, ovate bracts in a flat, rosette-like arrangement, deep-green ovate-shaped foliage with nearly no lobes, a medium-sized, v-shaped plant habit and a mid-season flowering response, is disclosed.

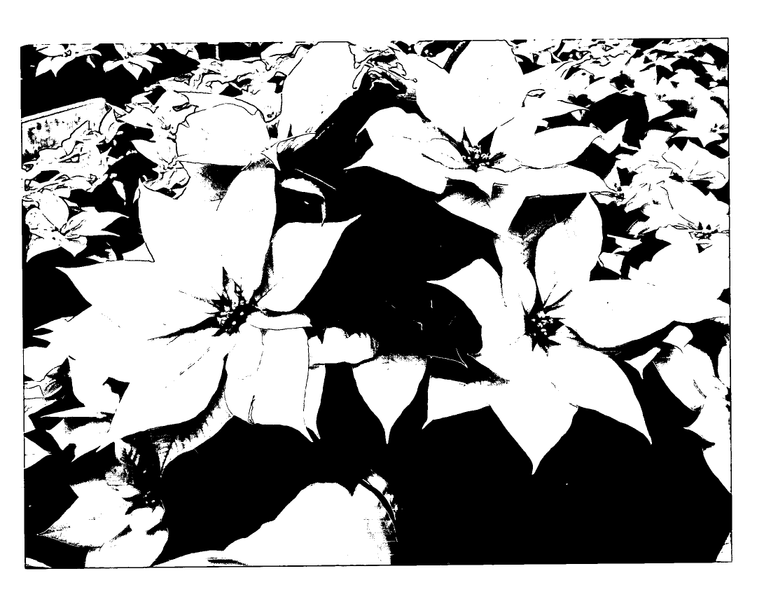

### GENUS AND SPECIES

Euphorbia pulcherrima (Wilid ex Klotzsch.)

### VARIETY DENOMINATION

‘Fismars Lipink’

### BACKGROUND OF THE NEW PLANT

The present invention relates to a new and distinct cultivar of poinsettia plant botanically known as Euphorbia pulcherrima (Willd.), and hereinafter referred to by the name ‘Fismars Lipink’. ‘Fismars Lipink’ is the result of a whole plant induced mutation in the commercial variety ‘Fismars’ (U.S. Plant Pat. No. 14,977), which is characterized by red-colored bracts, dark green foliage with only weak lobing and medium sized plant habit.

In May 2003, irradiations of young plants of ‘Fismars’ were conducted in Ahrensburg, Germany. The treated plants were grown in Hillscheid, Germany and were propagated by shoot tip cuttings. The resulting plants were screened for positive mutations and examined during the flowering period in winter 2003/2004. Among these plants, a single pink-flowered plant, m03-14-1, was selected and later asexually multiplied for further examinations in 2004. As a result, it was concluded that this new poinsettia plant is distinguishable from any other poinsettia variety whose existence is known to the inventor.

Horticultural examination of the plants starting in 2004 and continuing thereafter has confirmed that the combination of characteristics as herein disclosed for ‘Fismars Lipink’ are firmly fixed and retained through successive generations of asexual reproduction. ‘Fismars Lipink’ has been asexually reproduced by vegetative cuttings in Hillscheid, Germany over a two and one-half year period.

‘Fismars Lipink’ has not been made publicly available more than one year prior to the filing date of this application.

### SUMMARY OF THE INVENTION

The following traits have been repeatedly observed and are determined to be the basic characteristics of ‘Fismars Lipink’ which in combination distinguish this Poinsettia as a new and distinct cultivar when grown under normal horticultural practices in Hillscheid, Germany:
1. Light pink bracts;
2. Medium-sized, ovate bracts in a flat, rosette-like arrangement;
3. Deep-green, ovate-shaped foliage with nearly no lobes;
4. A medium sized, v-shaped plant habit; and
5. A mid-season flowering response.

### DESCRIPTION OF THE PHOTOGRAPH

This new Poinsettia plant is illustrated by the accompanying photograph which shows overall plant habit, including form, foliage and inflorescences. The colors shown are as true as can be reasonably obtained by conventional photographic procedures. The photograph is of a 19 week-old plant grown in Hillscheid, Germany in 2006.

### DESCRIPTION OF THE NEW CULTIVAR

‘Fismars Lipink’ has not been observed under all possible environmental conditions. The following observations, measurements and comparisons describe plants grown in Hillscheid, Germany under greenhouse conditions which approximate those generally used in commercial practice.

The plants described were grown in a greenhouse in Hillscheid, Germany, from the summer to winter of 2006. Rooted cuttings were planted in 14-cm pots on Jul. 26, 2006 and were pinched on Aug. 10, 2006 which left 7-8 leaves remaining. Initially, the minimum temperature was 18° C. Beginning Oct. 1, 2006 the temperature was lowered to a minimum temperature of 16° C. to 17° C. The plants initiated flowers under natural short-day conditions in the fall. No black cloth was applied to the greenhouse to simulate short-day conditions. No growth regulator was applied.

Observations and measurements were mainly taken in mid-December, when the plants were in full flower and about 20-weeks old. In the following description, color references are made to The Royal Horticultural Society Colour Chart (RHS), 2001. The color references were determined indoors in a north light.

### DESCRIPTION OF THE NEW PLANT

Classification:
   Family.—Euphorbiaceae.
   Botanical.—Euphorbia pulcherrima (Willd. ex Klotzsch).
   Common.—Poinsettia.
Parentage: Mutation of Poinsettia cultivar ‘Fismars’ (U.S. Plant Pat. No. 14,977)
Growth:
   Time to produce a rooted cutting.—20 to 24 days in a greenhouse at a temperature of 22° C. to 24° C.
   Blooming habit.—Beginning under natural short-day conditions in fall: Botanically (cyathia open): About December 1 Commercially (bracts colored, marketable): December 1 Flowering response time: About 10 weeks from equinox, total cultivation time depending on the plant size desired; for a branched plant in a 14-cm pot the flowering time is roughly 18 weeks Keeping quality: Good lasting quality is maintained for about 4 weeks with no dropping of bracts.
Plant:
   Form.—Shrub, self-branching.
   Growth habit.—Medium sized; moderately compact, pinched plants are bushy with the branches slanting upright at an angle of roughly 45°.
   Height.—23.0 cm as measured from the soil line to the top.
   Width.—40 cm to 52 cm.
   Number of branches.—8 to 11.
   Average number of inflorescences per plant.—7 to 9.
Stems:
   Color.—Lower part: RHS 143A to 143B (green) Upper part: Approximately RHS 146B (green) and weakly infused near the tips with RHS 176C (reddish).
   Length.—18 cm to 23 cm.
   Internode length.—1.5 cm to 3 cm.
   Diameter.—0.5 cm to 0.7 cm.
Leaves:
   Quantity.—55 to 65 leaves per plant (5-7 leaves per branch).
   Shape.—Ovate.
   Base.—Truncate to obtuse.
   Apex.—Acuminate.
   Lobes.—Hardly any, if occurring are very shallow.
   Margin.—Entire.
   Texture.—Upper surface: Smooth and flat, no rugosity, only weakly veined Lower surface: Flat and smooth, except for the slightly protruding midrib and finer side veins in a pinnate pattern.
   Vein color.—Upper surface: RHS 145C (green) Lower surface: RHS 145D (pale green).
   Size.—Length: 11 cm to 13.5 cm Width: 7 cm to 10 cm.
   Color.—Mature foliage: Upper surface: Approximately RHS 147A Lower surface: RHS 137D Immature foliage: Upper surface: RHS 143A Lower surface: RHS 137D.
   Leaf petiole.—Length: 4.5 cm to 8.5 cm Diameter: 0.3 cm Color: Upper surface: RHS 180C to 180D (light brown) in parts; other areas are RHS 146B Lower surface: RHS 145B to 145C (pale green) Aspect: Petioles are mostly horizontally directed and leaf blades slant somewhat downwards.
Inflorescence:
   Whole inflorescence with surrounding bracts.—Medium-sized, flat with the bracts overlapping and mostly horizontally directed; in the center a tight cyme with medium-sized cyathia.
   Diameter.—20 cm to 26 cm.
   Height of inflorescence.—2.5 cm to 3.5 cm.
Bracts:
   Number of bracts per inflorescence.—12 to 16.
   Shape.—Ovate.
   Base.—Obtuse (larger bracts) to acute (younger bracts).
   Apex.—Acuminate.
   Lobes.—Rounded, very weak to weak.
   Size.—Length: 11.5 cm to 12.0 cm Width: 6.5 cm to 8.2 cm.
   Texture.—mostly smooth, a little rugosity with younger bracts (not fully expanded).
   Vein color.—Upper surface: RHS 46D near base; other areas correspond closely to the bract color Lower surface: RHS 48A (reddish) near the base and fading towards the apex.
   Bract color.—Upper surface: Ranging from RHS 58D (largest and lowest fully colored bracts) to approximately RHS 52C (the true bracts forming the main whorl) and RHS 55A to even RHS 52B for the youngest, not yet fully expanded bracts Lower surface: RHS 52D.
   Bract petiole.—Length: 1.2 cm to 2.0 cm Color: Upper surface: RHS 46C (red) Lower surface: RHS 47A to RHS 50B or even lighter.
Cyme:
   Cyme diameter.—Approximately 1.8 cm to 2.3 cm.
   Cyathia number.—7 to 12 in a tight cluster.
   Cyathium.—Shape: Ovate Diameter: 0.5 cm to 0.6 cm Length: 0.6 cm to 0.7 cm Color: Mainly RHS 143B (green); top is RHS 45D (light red).
   Peduncle.—Color: RHS 143C (light green) Length: 0.02 cm to 0.03 cm.
   Nectar cups.—Number: Usually only one per cyathium Size: Up to 0.6 cm wide Color: RHS 15A (yellow-orange), a little RHS N30A (reddish) color may occur at the margin.
Reproductive organs:
   Stamens.—Strap-like Quantity: About 15 to 20 in a cluster Filaments: RHS 46B (red) Length: 0.3 cm Pollen: RHS 13B (yellow) Female flowers/pistils have not been observed.
Fruit and seed set: Has not been observed.
Disease and insect resistance: No observations made

### COMPARISON WITH KNOWN CULTIVARS

The most similar cultivars in comparison to ‘Fismars Lipink’ are the parental cultivar ‘Fismars’ (U.S. Plant Pat. No. 14,977), ‘Fismars Pink’ (U.S. Plant Pat. No. 18,866) and the commercial cultivars ‘Fiscorosa’ (U.S. Plant Pat. No. 10,077) and ‘Eckalbert’ (U.S. Plant patent application Ser. No. 10/291,043).

In comparison to the parent ‘Fismars’ (U.S. Plant Pat. No. 14,977), ‘Fismars Lipink’ differs from the parental cultivar in that ‘Fismars Lipink’ has pink bracts, while ‘Fismars’ has red bracts.

In comparison to ‘Fismars Pink’ (U.S. Plant Patent application applied for), ‘Fismars Lipink’ has a somewhat lighter overall bract color, inflorescences with more cyathia and a somewhat taller plant habit than ‘Fismars Pink’. Additionally, ‘Fismars Lipink’ has a slightly later flowering response (mid-season) in the fall than ‘Fismars Pink’ (early to mid-season).

In comparison to ‘Fiscorosa’ (U.S. Plant Pat. No. 10,077, ‘Fismars Lipink’ has smaller bracts and leaves with nearly no lobes. Additionally, ‘Fiscorosa’ has a taller and wider plant habit than ‘Fismars Lipink.

In comparison to ‘Eckalbert (U.S. Plant patent application Ser. No. 10/291,043), ‘Eckalbert’ has funnel-shaped inflorescences, while the inflorescences of ‘Fismars Lipink’ are flatter and have shorter bracts that are mostly horizontally directed. Additionally, the leaves and bracts of ‘Fismars Lipink’ develop nearly no lobes, while the leaves and bracts of ‘Eckalbert’ are moderately oak-leaf shaped.

I claim:

1. A new and distinct cultivar of Poinsettia plant as shown and described herein.
